(19) United States
(12) Patent Application Publication
(10) Pub. No.: US 2022/0033813 A1
(43) Pub. Date: Feb. 3, 2022

(54) L-OLIGONUCLEOTIDE INHIBITORS OF POLYCOMB REPRESSIVE COMPLEX 2 (PRC2)

(71) Applicant: THE TEXAS A&M UNIVERSITY SYSTEM, College Station, TX (US)

(72) Inventors: Jonathan T. Sczepanski, College Station, TX (US); Charles E. Deckard, College Station, TX (US)

(21) Appl. No.: 17/275,132

(22) PCT Filed: Sep. 5, 2019
(86) PCT No.: PCT/US2019/049759

§ 371 Date: Mar. 10, 2021

Related U.S. Application Data
(60) Provisional application No. 62/729,743, filed on Sep. 11, 2018.

(51) Int. Cl. C12N 15/113; A61K 31/712

(52) U.S. Cl. CPC C12N 15/113; A61K 31/712; C12N 2310/3231; C12N 2310/50; C12N 2320/30

(57) ABSTRACT

The present invention contemplates a method for the treatment of cancer comprising L-nucieic acid. The present invention contemplates a method for the treatment of cancer comprising guanosine-rich L-oligonucleotides. The present invention also relates to Polycomb Repressive Complex 2 (PRC 2) 1, -oligonucleotide inhibitors and their use for the treatment of cancer and other conditions associated with aberrant PRC2 methyl transferase activity.

### CROSS-REFERENCE TO RELATED APPLICATIONS

The present application claims the benefit of U.S. Provisional Patent Application No. 62/729,743, filed on Sep. 11, 2018, which is incorporated herein by reference.

### FIELD OF THE INVENTION

The present invention contemplates a method for the treatment of cancer comprising L-nucleic acid. The present invention contemplates a method for the treatment of cancer comprising guanosine-rich L-oligonucleotides. The present invention also relates to Polycomb Repressive Complex 2 (PRC2) L-oligonucleotide inhibitors and their use for the treatment of cancer and other conditions associated with aberrant PRC2 methyltransferase activity.

### BACKGROUND OF THE INVENTION

Post-translational modifications of the amino-terminal ‘tail’ (as well other non-tail sites) of histone H3 are critical for multiple DNA-templated processes. Notably, H3K27 is the target of methylation by Polycomb Repressive Complex 2 (PRC2) to modulate gene transcription (and in some cases, acetylation, brought about by distinct enzyme systems. The mono-, di-, and tri-methylation states of histone H3-K27 are associated with different functions in transcriptional control. Histone H3-K27 monomethylation (or acetylation) is often associated with active transcription of genes, such as differentiation genes, that are poised for transcription (Cui et al. “Chromatin Signatures in Multipotent Human Hematopoietic Stem Cells Indicate the Fate of Bivalent Genes During Differentiation, Cell Stem Cell 4:80-93 (2009) [1] and Barski et al., “High-Resolution Profiling of Histone Methylation in the Human Genome,” Cell 129:823-37 (2007) [2]). In contrast, trimethylation of histone H3-K27 is largely associated with either transcriptionally repressed genes or genes that are poised for transcription when histone H3-K4 trimethylation is in cis (Cui et al. “Chromatin Signatures in Multipotent Human Hematopoietic Stern Cells Indicate the Fate of Bivalent Genes During Differentiation, Cell Stem Cell 4:80-93 (2009) [1]; Kirmizis et al. “Silencing of Human Polycomb Target Genes is Associated with Methylation of Histone H3 Lys 27,” Genes Dev 18:1592-1605 (2007) [3]; Bernstein et al. “A Bivalent Chromatin Structure Marks Key Developmental Genes in Embryonic Stem Cells,” Cell 125:315-26 (2006) [4].

The overexpression of genes in the PRC2 complex has been associated with a number of cancers, including, for example, metastatic prostate cancer (Crea et al., “Pharmacologic Disruption of Polycomb Repressive Complex 2 Inhibits Tumorigenicity and Tumor Progression in Prostate Cancer,” Mol. Cancer 10:40 (2011) [5], breast cancer (Holm, K. et al. (2012) “Global H3k27 Trimethylation and Ezh2 Abundance in Breast Tumor Subtypes,” Mol. Oncol. 6(5), 494-506. [6]), bladder cancer (Raman et al., “Increased Expression of the Polycomb Group Gene, EZH2, in Transitional Cell Carcinoma of the Bladder,” Clin. Cancer Res. 11:8570-6 (2005) [7]), gastric cancer (Matsukawa et al., “Expression of the Enhancer of Zeste Homolog 2 is Correlated with Poor Prognosis in Human Gastric Cancer,” Cancer Sci. 97:484-91 (2006) [8]), melanoma, and lymphoma (McCabe et al,, “Mutation of A677 in Histone Methyltransferase EZH2 in Human B-cell Lymphoma Promotes Hypertrimethylation of Histone H3 on Lysine 27 (H3K27),” Proc. Nat'l Acad. Sci. USA 109(8):2989-94 (2012) [9]). The overexpression of polycomb genes and subsequent increase in PRC2 complex activity that has been reported in cancer is predicted to increase the trimethylated state of histone H3-K27 and thus result in transcriptional repression of several tumor suppressor genes (Crea et al., “EZH2 Inhibition: Targeting the Crossroad of Tumor Invasion and Angiogenesis,” Cancer Metastasis Rev. 31(3-4), 753-761. (2012) [10]. Accordingly, agents capable of disrupting this cascade of events would be therapeutically useful for the treatment of cancer.

### SUMMARY OF THE INVENTION

The present invention contemplates a method for the treatment of cancer comprising L-nucleic acid. The present invention contemplates a method for the treatment of cancer comprising guanosine-rich L-oligonucleotides. The present invention also relates to Polycomb Repressive Complex 2 (PRC2) L-oligonucleotide inhibitors and their use for the treatment of cancer and other conditions associated with aberrant PRC2 methyltransferase activity.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Other objects, advantages, and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

In one embodiment, the invention contemplates a method of treatment of a subject with cancer comprising: (a) providing: (i) L-nucleic acid, and (b) treating said subject with said L-nucleic acid. In one embodiment, said L-nucleic acid comprises a guanosine-rich L-oligonucleotide. In one embodiment, said guanosine-rich L-oligonucleotide comprises at least 1 GGN motif. In one embodiment, said guanosine-rich L-oligonucleotide comprises 4-100 nucleotides. In one embodiment, said guanosine-rich L-oligonucleotide forms G-quartets. In one embodiment, said L-nucleic acid comprises a non-guanosine-rich L-oligonucleotide. In one embodiment, said L-nucleic acid further comprises a chemical modification. In one embodiment, said L-nucleic acid comprises β-L-nucleic acid. In one embodiment, said L-nucleic acid comprises a guanosine-rich L-oligonucleotide. In one embodiment, said L-nucleic acid comprises L-ribose nucleic acid. In one embodiment, said L-ribose nucleic acid comprises L-[GGAA]₁₀ [SEQ ID NO: 1]. In one embodiment, said L-ribose nucleic acid comprises 5′-AA-GGAA-GGAA-GGAA-GGAA-GGAA-GGAA-GGAA-GGAA-GGAA-GGAA [SEQ ID NO: 2]. In one embodiment, said L-ribose nucleic acid comprises L-[G3A4]₄ [SEQ ID NO: 3]. In one embodiment, said L-ribose nucleic acid comprises 5′-AA-AAA-GGGAAAA-GGGAAAA-GGGAAAA-GGAAAA [SEQ ID NO: 4]. In one embodiment, said L-ribose nucleic acid comprises a G-quadruplex forming L-RNA. In one embodiment, said L-nucleic acid comprises L-deoxyribose nucleic acid. In one embodiment, said L-deoxyribose nucleic acid comprises L-[GGAA]₁₀ [SEQ ID NO: 5]. In one embodiment, said L-deoxyribose nucleic acid comprises 5′-AA-GGAA-GGAA-GGAA-GGAA-GGAA-GGAA-GGAA-GGAA-GGAA-GGAA [SEQ ID NO: 6]. In one embodiment, said L-deoxyribose nucleic acid comprises L-[G3A4]₄ [SEQ ID NO: 7]. In one embodiment, said L-deoxyribose nucleic acid comprises 5′-AA-AAA-GGGAAAA-GGGAAAA-GGGAAAA-GGAAAA [SEQ ID NO: 8]. In one embodiment, said L-nucleic acid inhibits PRC2.

In one embodiment, the invention contemplates a method of inhibiting PRC2 in a subject comprising: (a) providing: (i) L-nucleic acid, and (b) treating said subject with said L-nucleic acid. In one embodiment, said L-nucleic acid comprises a guanosine-rich L-oligonucleotide. In one embodiment, said guanosine-rich L-oligonucleotide comprises at least 1 GGN motif. In one embodiment, said guanosine-rich L-oligonucleotide comprises 4-100 nucleotides. In one embodiment, said guanosine-rich L-oligonucleotide forms G-quartets. In one embodiment, said L-nucleic acid comprises a non-guanosine-rich L-oligonucleotide. In one embodiment, said L-nucleic acid further comprises a chemical modification. In one embodiment, said L-nucleic acid comprises β-L-nucleic acid. In one embodiment, said L-nucleic acid comprises a guanosine-rich L-oligonucleotide. In one embodiment, said L-nucleic acid comprises L-ribose nucleic acid. In one embodiment, said L-ribose nucleic acid comprises L-[GGAA]₁₀ [SEQ ID NO: 9]. In one embodiment, said L-ribose nucleic acid comprises 5′-AA-GGAA-GGAA-GGAA-GGAA-GGAA-GGAA-GGAA-GGAA-GGAA-GGAA [SEQ ID NO: 10]. In one embodiment, said L-ribose nucleic acid comprises L-[G3A4]₄ [SEQ ID NO: 11]. In one embodiment, said L-ribose nucleic acid comprises 5′-AA-AAA-GGGAAAA-GGGAAAA-GGGAAAA-GGAAAA [SEQ ID NO: 12]. In one embodiment, said L-ribose nucleic acid comprises a G-quadruplex forming L-RNA. In one embodiment, said L-nucleic acid comprises L-deoxyribose nucleic acid. In one embodiment, said L-deoxyribose nucleic acid comprises L-[GGAA]₁₀ [SEQ ID NO: 13]. In one embodiment, said L-deoxyribose nucleic acid comprises 5′-AA-GGAA-GGAA-GGAA-GGAA-GGAA-GGAA-GGAA-GGAA-GGAA-GGAA [SEQ ID NO: 14]. In one embodiment, said L-deoxyribose nucleic acid comprises L-[G3A4]₄ [SEQ ID NO: 15]. In one embodiment, said L-deoxyribose nucleic acid comprises 5′-AA-AAA-GGGAAAA-GGGAAAA-GGGAAAA-GGAAAA [SEQ ID NO: 16].

In one embodiment, the invention contemplates a method of treating cancer, comprising: a) providing i) a subject with cancer, said cancer overexpressing PRC2 and ii) a composition comprising L-nucleic acids; and b) administering said composition to said subject. In one embodiment, said cancer exhibits resistance to SAM-competitive inhibitors. In one embodiment, said L-nucleic acid inhibits PRC2. In one embodiment, said L-nucleic acid comprises a guanosine-rich L-oligonucleotide. In one embodiment, said guanosine-rich L-oligonucleotide comprises at least 1 GGN motif. In one embodiment, said guanosine-rich L-oligonucleotide comprises 4-100 nucleotides. In one embodiment, said guanosine-rich L-oligonucleotide forms G-quartets. In one embodiment, said L-nucleic acid further comprises a chemical modification. In one embodiment, said L-nucleic acid comprises β-L-nucleic acid. In one embodiment, said L-nucleic acid comprises a non-guanosine-rich L-oligonucleotide. In one embodiment, said L-nucleic acid comprises L-ribose nucleic acid. In one embodiment, said L-ribose nucleic acid comprises L-[GGAA]₁₀. In one embodiment, said L-ribose nucleic acid comprises SEQ ID NO: 2. In one embodiment, said L-ribose nucleic acid comprises L-[G3A4]₄. In one embodiment, said L-ribose nucleic acid comprises SEQ ID NO: 4. In one embodiment, said L-ribose nucleic acid comprises a G-quadruplex forming L-RNA. In one embodiment, said L-nucleic acid comprises L-deoxyribose nucleic acid. In one embodiment, said L-deoxyribose nucleic acid comprises L-[GGAA]₁₀. In one embodiment, said L-deoxyribose nucleic acid comprises L-[G3A4]₄. In one embodiment, said L-deoxyribose nucleic acid comprises SEQ ID NO: 6. In one embodiment, said L-deoxyribose nucleic acid comprises SEQ ID NO: 8.

In one embodiment, the invention contemplates a method of screening, comprising a) providing cancer cells ex vivo, said cancer cells overexpressing PRC2 and ii) at least two different L-nucleic acids; and b) testing said at least two different L-nucleic acids for inhibition of PRC2 by exposing said cancer cells to said L-nucleic acids. In one embodiment, said cancer cells exhibit resistance to SAM-competitive inhibitors. In one embodiment, at least one of said L-nucleic acids comprises a guanosine-rich L-oligonucleotide. In one embodiment, said guanosine-rich L-oligonucleotide comprises at least 1 GGN motif. In one embodiment, said guanosine-rich L-oligonucleotide comprises 4-100 nucleotides. In one embodiment, said guanosine-rich L-oligonucleotide forms G-quartets. In one embodiment, at least one of said L-nucleic acids further comprises a chemical modification. In one embodiment, at least one of said L-nucleic acids comprises β-L-nucleic acid. In one embodiment, at least one of said L-nucleic acids comprises a non-guanosine-rich L-oligonucleotide. In one embodiment, at least one of said L-nucleic acids comprises a L-ribose nucleic acid. In one embodiment, said L-ribose nucleic acid comprises L-[GGAA]₁₀. In one embodiment, said L-ribose nucleic acid comprises SEQ ID NO: 2. In one embodiment, said L-ribose nucleic acid comprises L-[G3A4]₄. In one embodiment, said L-ribose nucleic acid comprises SEQ ID NO: 4. In one embodiment, said L-ribose nucleic acid comprises a G-quadruplex forming L-RNA. In one embodiment, at least one of said L-nucleic acids comprises a L-deoxyribose nucleic acid. In one embodiment, said L-deoxyribose nucleic acid comprises L-[GGAA]₁₀. In one embodiment, said L-deoxyribose nucleic acid comprises L-[G3A4]₄. In one embodiment, said L-deoxyribose nucleic acid comprises SEQ ID NO: 6. In one embodiment, said L-deoxyribose nucleic acid comprises SEQ ID NO: 8.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “polycomb repressive complex 2,” commonly abbreviated to PRC2, as used herein describes one of the two classes of polycomb-group proteins or (PcG). The other component of this group of proteins is PRC1 (Polycomb Repressive Complex 1). This complex has histone methyltransferase activity and primarily trimethylates histone H3 on lysine 27 (i.e. H3K27me3) [11, 12], a mark of transcriptionally silent chromatin. PRC2 is required for initial targeting of genomic region (PRC Response Elements or PRE) to be silenced, while PRC1 is required for stabilizing this silencing and underlies cellular memory of silenced region after cellular differentiation. PRC1 also mono-ubiquitinates histone H2A on lysine 119 (H2AK119Ub1). These proteins are required for long term epigenetic silencing of chromatin and have an important role in stem cell differentiation and early embryonic development. PRC2 are present in all multicellular organisms. PRC2 has a role in X chromosome inactivation, in maintenance of stern cell fate, and in imprinting. Aberrant expression of PRC2 has been observed in cancer [11, 12]. The PRC2 is evolutionarily conserved, and has been found in mammals, insects, and plants.

The term “in vivo imaging” as used herein refers to those techniques that non-invasively produce images of all or part of an internal aspect of a mammalian subject.

By the term “biological targeting moiety” (BTM) is meant a compound which, after administration, is taken up selectively or localizes at a particular site of the mammalian body in vivo. Such sites may be implicated in a particular disease state or be indicative of how an organ or metabolic process is functioning.

By the term “L-nucleic acid” is meant L-nucleic acid or L-nucleic acid analogue (e.g. modified nucleobase) which may be of purely synthetic origin, and may be optically pure, i.e. a single enantiomer and hence chiral. Conventional 1-letter or single letter abbreviations for L-nucleic acid are used herein. L-nucleic acid can be L-ribonucleic (L-RNA) or L-deoxyribonucleic (L-DNA) or analogues thereof.

By the term “L-ribonucleic acid aptamer” is an RNA-like molecule built from L-ribose units [13]. It is an artificial oligonucleotide named for being a mirror image of natural oligonucleotides. Due to their L-nucleotides, it is believed that they are highly resistant to degradation by nucleases [14].

By the term “L-deoxyribonucleic acid” or “L-DNA aptamer”, is an DNA-like molecule built from L-deoxyribose units. It is an artificial oligonucleotide named for being a mirror image of natural oligonucleotides. Due to their L-nucleotides, it is believed that they are highly resistant to degradation by nucleases.

By the term “amino acid” is meant an L- or D-amino acid, amino acid analogue (eg. naphthylalanine) which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure.

By the term “GGN motif” is meant a repeated GGN trinucleotide within a sequence.

By the term “G-quartets” is meant secondary structures [15] are formed in nucleic acids by sequences that are rich in guanine. They are helical structures containing guanine tetrads that can form from one, two or four strands. The unimolecular forms often occur naturally near the ends of the chromosomes, better known as the telomeric regions, and in transcriptional regulatory regions of multiple genes and oncogenes [16, 17]. Four guanine bases can associate through Hoogsteen hydrogen bonding to form a square planar structure called a guanine tetrad (also called G-tetrad or G-quartet), and two or more guanine tetrads can stack on top of each other to form a G-quadruplex.

By the term “G-quadruplex” is meant secondary structures are formed in nucleic acids by sequences that are rich in guanine. The length of the nucleic acid sequences involved in formation determines how the quadruplex folds. Short sequences, consisting of only a single contiguous run of three or more guanine bases, require four individual strands to form a quadruplex. Such a quadruplex is described as tetramolecular, reflecting the requirement of four separate strands. The term G4 DNA was originally reserved for these tetramolecular structures that might play a role in meiosis. However, as currently used in molecular biology, the term G4 can mean G-quadruplexes of any molecularity. Longer sequences, which contain two contiguous runs of three or more guanine bases, where the guanine regions are separated by one or more bases, only require two such sequences to provide enough guanine bases to form a quadruplex. These structures, formed from two separate G-rich strands, are termed bimolecular quadruplexes. Finally, sequences which contain four distinct runs of guanine bases can form stable quadruplex structures by themselves, and a quadruplex formed entirely from a single strand is called an intramolecular quadruplex [18].

By the phrase “in a form suitable for mammalian administration” is meant a composition which is sterile, pyrogen-free, lacks compounds which produce toxic or adverse effects, and is formulated at a biocompatible pH (approximately pH 4.0 to 10.5). Such compositions lack particulates which could risk causing emboli in vivo, and are formulated so that precipitation does not occur on contact with biological fluids (e.g. blood). Such compositions also contain only biologically compatible excipients, and are preferably isotonic.

The “biocompatible carrier” is a fluid, especially a liquid, in which the an agent can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer.

Preferred multiple dose containers comprise a single bulk vial (e.g. of 10 to 50 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may contain additional optional excipients such as: an antimicrobial preservative, pH-adjusting agent, filler, radioprotectant, solubiliser or osmolality adjusting agent.

By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The term “protected” refers to the use of a protecting group. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. For example: amine protecting groups are well known to those skilled in the art and are suitably chosen from: Boc (where Boc is tert-butyloxycarbonyl); Eei (where Eei is ethoxyethylidene); Fmoc (where Fmoc is fluorenylmethoxycarbonyl); trifluoroacetyl; allyloxycarbonyl; Dde [i.e. 1-(4,4-dimethyl-2,6-dioxocyclohexylidene)ethyl] or Npys (i.e. 3-nitro-2-pyridine sulfenyl). The use of further protecting groups are described in Protective Groups in Organic Synthesis, 4^{th} Edition, Theorodora W. Greene and Peter G. M. Wuts, (2006) [19].

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1A-C shows PRC2 binds G4 RNA irrespective of stereochemistry. FIG. 1a show a CD spectra of D- and L-(GGAA)₁₀ RNAs. FIG. 1b shows representative EMSA gels (1% agarose) of (GGAA)₁₀ binding to PRC2 (0-1 μM). Binding mixtures contained 2 nM Cy3-labeled (GGAA)₁₀, 100 mM KCl, 2.5 mM MgCl₂, 0.1 mM ZnCl₂, 2 mM BME, 0.1 mg/mL BSA, 5% glycerol, and 50 mM TRIS-HCl pH 7.5. FIG. 1c shows a saturation plot for binding of either D- (GGAA)₁₀ or L-(GGAA)₁₀ to PRC2. Error bars show SD (n=3).

FIGS. 2A&B shows both enantiomers of (GGAA)₁₀ bind to the same site on PRC2. Pre-formed PRC2-D-(GGAA)₁₀ complexes are disrupted by L-(GGAA)₁₀ (FIG. 2A) and vice versa (FIG. 2B). Initial PRC2-RNA complexes were prepared using the binding conditions described in FIG. 1b except constant concentrations of PRC2 (100 nM) and Cy3-Labled (GGAA)₁₀ RNA (10 nM) were used. Data points represent a 2-fold titration of the unlabeled competitor ranging from 0.01-1.3 μM.

FIG. 3 shows L-(GGAA)₁₀ outcompetes native substrates for binding to PRC2.

FIG. 3a shows pre-formed complexes between PRC2 (250 nM) and HOTAIR-300 (25 nM) are disrupted by L-(GGAA)₁₀.

FIG. 3b shows pre-formed complexes between PRC2 (1000 nM) and a 12-mer oligonucleosome array (8 nM) are disrupted by L-(GGAA)₁₀. Initial PRC2-substrate complexes in both panels were prepared using the binding conditions described in FIG. 1b except where indicated (above). Data points represent a 3-fold titration of unlabeled L-(GGAA)₁₀ ranging from 0.001-3 μM.

FIG. 4A-C shows Oligonucleotides used in this work.

FIG. 4A Sequences of oligonucleotides used for binding EMSAs and competition experiments. Terminal D-deoxyribose residues (D-dA) on the RNA strands are underlined.

FIG. 4B shows a denaturing PAGE analysis of the Cy3-labeled oligonucleotides presented in FIG. 4A (10%, 29:1 acrylamide:bisacrylamide).

FIG. 4C shows a native PAGE analysis (10%, 29:1 acrylamide:bisacrylamide) of the same oligonucleotides in FIG. 4B. The running buffer (1×TBE) was supplemented with 10 mM KOAc. The increased electrophoretic mobility of (GGAA)₁₀ and (G3A4)₄ relative to (A)₄₀ is indicative of the G4 structure formation by these oligonucleotides in the presence of K+.

FIG. 5A-C shows that PRC2 binds both D- and L-(GGAA)₁₀ RNAs in the presence Li+.

FIG. 5A shows a CD spectra of D- and L-(GGAA)₁₀ in the presence of either 100 mM KCl or LiCl.

FIG. 5B shows representative EMSA gels (1% agarose, 0.2×TBE supplemented with 10 mM LiOAc) of (GGAA)10 binding to PRC2 (0-1 μM) in the presence of Li+. Binding conditions were the same as described in FIG. 1b (main text), except that the KCl was replaced with LiCl.

FIG. 5C shows a saturation plot for binding of either D- or L-(GGAA)₁₀ to PRC2 in the presence of Li+. Error bars show SD (n32 3).

FIG. 6A-C shows PRC2 binds similarly to both D- and L-(G3A4)₄ G4 RNAs.

FIG. 6A shows a CD spectra of D- and L-(G3A4)₄.

FIG. 6B shows a representative EMSA gels (1% agarose, 0.2×TBE supplemented with 10 mM KOAc) of (GGAA)₁₀ binding to PRC2 (0-1 μM). Binding conditions were the same as described in FIG. 1b (main text).

FIG. 6c shows a saturation plot for binding of either D- or L-(G3A4)₄ to PRC2. Error bars show SD (n=3).

FIG. 7A-D shows PRC2 binds weakly to (A)₄₀ and D-(dGGAA)₁₀.

FIGS. 7A&B shows a CD spectra of both enantiomers of (A)₄₀ and the D-(dGGAA)₁₀, respectively.

FIG. 7c shows a representative EMSA gels (1% agarose, 0.2×TBE supplemented with 10 mM KOAc) of D-(A)₄₀, L-(A)₄₀, and D-(dGGAA)₁₀ binding to PRC2 (0-2 μM). Binding conditions were the same as described in FIG. 1b (main text).

FIG. 7d shows a saturation plot for binding of D-(A)₄₀, L-(A)₄₀, and D-(dGGAA)₁₀ to PRC2. Error bars show SD (n=3).

FIG. 8 shows a competitive binding experiments for L-(A)₄₀ versus pre-formed PRC2-(GGAA)₁₀ complexes. Initial PRC2-RNA complexes were prepared using the binding conditions described in FIG. 1b, and competitor (A)₄₀ RNA was added in 2-fold increments from 80 nM to 10 uM.

FIG. 9A-C shows Disruption of PRC2-HOTAIR complexes.

FIG. 9A shows an EMSA gel (1% agarose, 0.2×TBE supplemented with 10 mM KOAc) of PRC2 (0.1-2000 nM) binding Cy5-labeled HOTAIR-300 (25 nM). Binding conditions were the same as described in FIG. 1b (main text).

FIG. 9B shows that D-(GGAA)₁₀ is able to outcompete HOTAIR-300 for binding to PRC2. Initial PRC2-HOTAIR complexes were prepared using the binding conditions described in FIG. 1b (main text) except where indicated (above). Data points represent a 3-fold titration of unlabeled D-(GGAA)₁₀ ranging from 0.001-3 μM.

FIG. 9C shows that L-(A)₄₀ is unable to compete with HOTAIR for binding to PRC2.

FIG. 10A-E shows 12-mer oligonucleosome array assembly.

FIG. 10A shows a schematic of Cy5-labeled 12-mer oligonucleosome array employed in the PRC2 binding and competition assays. A single Cy5 dye is positioned within the fifth nucleosome unit (N5).

FIG. 10B shows an insertion of the Cy5 dye containing oligonucleotide was confirmed by 10% native PAGE (29:1, acrylamide:bisacrylamide). Lane 1, ladder; lane 2, unmodified N5 DNA fragment; lane 3, nicked N5 DNA fragment; lane 4, Cy5-labeled N5 DNA fragment following the strand exchange process.

FIG. 10C shows reconstitution of Cy5-labeled oligonucleosome arrays. Agarose gel (0.6%, 0.2×TBE) analysis of Mg2+-induced precipitation of reconstitutions for several histone octamer:DNA ratios visualized with different fluorescent channels (see figure heading). Aliquots from re-suspended nucleosome pellets (P) and the supernatant (S) following Mg2+ precipitation are indicated for each octamer:DNA ratio employed.

FIG. 10D shows a restriction enzyme digest analysis of the Cy5-containing N5 fragment (5%, 59:1 acrylamide:bisacrylamide). Both naked (DNA) and reconstituted (Nuc) 12-mer arrays were digested similarly and their corresponding N5 fragments analyzed side-by-side.

FIG. 10E shows an EMSA gel (0.5% agarose, 0.2×TBE supplemented with 10 mM KOAc) of PRC2 (0.1-2000 nM) binding to the Cy5-labled array (8 nM). Binding conditions were the same as described in FIG. 1b (main text) except where indicated.

FIG. 11 shows is an illustration of the chirality independent nature of nucleotide binding to PRC2.

### DETAILED DESCRIPTION OF THE INVENTION

The present invention contemplates a method for the treatment of cancer comprising L-nucleic acid. The present invention contemplates a method for the treatment of cancer comprising guanosine-rich L-oligonucleotides. The present invention also relates to Polycomb Repressive Complex 2 (PRC2) L-oligonucleotide inhibitors and their use for the treatment of cancer and other conditions associated with aberrant PRC2 methyltransferase activity.

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout the specification may, but do not necessarily, refer to the same embodiment.

Furthermore, the described features, advantages and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment”, “an embodiment”, or similar language means that a particular feature, structure, or characteristic described in connection with the indicated embodiment is included in at least one embodiment of the present invention. Thus, the phrases “in one embodiment”, “in an embodiment”, and similar language throughout this specification may, but do not necessarily, all refer to the same embodiment.

The Polycomb Repressive Complex 2 (PRC2) is a multimeric protein complex constituted by four core proteins: RBBP7/1, SUZ12, EED, and the histone methyltransferase subunit, EZH1/2, that catalyzes the mono-, di- and tri-methylation of H3K27 (H3K27me/me²/me³). H3K27me³ is established exclusively by PRC2 and is an epigenetic mark associated with gene silencing. The repressive activity of PRC2 is critical for the proper regulation of lineage specific genes as well as X-inactivation. Furthermore, dysregulation of PRC2 and its epigenetic mark has been linked to numerous types of cancers, including prostate and breast cancers, non-Hodgkin lymphoma, and leukemia. The involvement of PRC2 in a multitude of cancers has motivated the development of small molecule PRC2 inhibitors, several of which are currently undergoing clinical trials.

Recently, long non-coding (lnc)RNAs have emerged as integral partners of PRC2, acting as molecular scaffolds capable of recruiting and maintaining PRC2 near target genes. Quantitative binding studies have identified that RNA G-quadruplex structures are bound preferentially over all other motifs. Importantly, binding of PRC2 by G-quadruplex structures was shown to inhibit the methyltransferase activity of the enzyme, providing an exciting opportunity to develop novel RNA-based inhibitors of PRC2. Indeed, the invention exploits this property by targeting PCR2 using L-(deoxy)ribose nucleic acids (L-DNA and L-RNA), which are bio-inert enantiomers (or mirror-images) of native D-nucleic acids. Specifically, herein it is demonstrated that PRC2 binds G-quadruplex RNAs irrespective of their stereochemistry. Moreover, it has been observed that several synthetic L-RNA G-quadruplexes (e.g. L-[GGAA]₁₀) can prevent PRC2 from binding its endogenous substrates, including chromatin and long noncoding RNAs. Most importantly, herein it is shown that G-quadruplexe-forming L-RNAs are potent inhibitors of PRC2 methyltransferase activity. This unprecedented discovery may open the door for the development of a novel class of PRC2 inhibitors for anticancer therapy.

In one embodiment, the invention comprises β-L-(deoxy)ribose nucleic acids (β-L-DNA, β-L-RNA, and derivatives thereof) that are capable of binding and inhibiting PRC2 (or any combination of its subunits). These include guanosine-rich L-oligonucleotides (GRLOs), and more specifically, G-quadruplex forming L-RNAs. Characteristics of GRLOs include: (1) having at least 1 GGN motif, (2) preferably having 4-100 nucleotides, although GRLOs having many more nucleotides are possible, and (3) optionally having chemical modifications. Especially useful GRLOs form G-quartets (and higher-order G-quadruplex structures), as indicated by a reversible and diagnostic thermal denaturation/renaturation profile at 295 nm. In one embodiment, preferred GRLOs also compete with their enantiomers, as well as a native D-[GGAA]₁₀ oligonucleotide, for binding to PRC2 (or any combination of its subunits) in an electrophoretic mobility shift assay.

The vast majority of PCR2 inhibitors are small molecules, including all compounds currently undergoing clinical trials. The invention described here is unique because it is an oligonucleotide-based inhibitor of PRC2 and represents a novel class of anticancer agents. Moreover, the invention is unique in the context of therapeutic oligonucleotides because it is comprised of mirror-image L-nucleic acids, which are completely orthogonal to the stereospecific environment of the cell (i.e. L-oligonucleotides are resistant to both nuclease degradation and off-target interactions with cellular components). These properties make L-oligonucleotides ideal therepeutic reagents, potentially as potential anticancer therapeutics.

The invention will be used to develop a novel anticancer therapeutic targeting PRC2. Overexpression of PRC2 is observed in a variety of human cancers and is linked to proliferation and poor prognosis. As a result, several small molecule inhibitors of PRC2 or its subunits (e.g. EZH2 and EED) have been developed, including several compounds currently undergoing clinical trials. Inhibition of either normal or hyperactive PRC2 has been shown to decrease cell survival and tumour growth in several types of cancer, which highlights the potential benefits of PRC2 inhibitors for the treatment of these cancers.

The vast majority of PCR2 inhibitors are small molecules, including all compounds currently undergoing clinical trials. Other potential inhibitory approaches include the use of peptidomimetics, antisense oligonucleotides, and RNAi. However, these latter approaches have not been applied clinically.

The invention provides several advantages as compared to current PRC2 inhibitors:
(1) L-oligonucleotides are chemically stable, nontoxic, and do not elicit an immune response.
(2) Unlike many of the current small molecule-based inhibitors of PRC2, which compete with the cofactor S-adenosylmethionine (SAM) and are susceptible to drug resistance, the L-oligonucleotide-based inhibitors described herein bind at an allosteric site, thereby overcoming common resistance mechanisms. Consequently, the invention has a potential therapeutic advantage for treating cancers with acquired resistance to traditional SAM-competitive inhibitors.
(3) Oligonucleotide-based therapeutics, including the inhibitors described here, can be easily conjugated to a variety of cell-specific moeities for targeted cancer therapy. This approach is extremely difficult for small molecule-based inhibitors due to the lack of general synthetic methods.
(4) Hybridization-based antidotes can be easily developed against the PRC2 inhibitors presented herein. Antidote control is the safest way to regulate drug activity because it is independent of underlying patient physiology and co-morbidities. In contrast, it is very difficult to generate antidotes for small molecules.
(5) Unlike small molecules, oligonucleotides are evolvable. Therefore, more potent or completely novel versions of these L-oligonucleotide inhibitors can be easily obtained using standard laboratory techniques. This also makes these inhibitors highly adaptable toward drug resistance mechanisms. In contrast, small molecule optimization and discovery is often time consuming and requires methods and/or equipment that is not readily accessible, which severely limits adaptability.

### Introduction

The Polycomb Repressive Complex 2 (PRC2) interacts promiscuously with G-quadruplex (G4) RNA structures. Herein, the limit of this promiscuity was tested by exploring the interaction of PRC2 with G4 RNAs comprised of L-ribonucleic acids (L-RNA), the enantiomer of naturally occurring D-RNA. Remarkably, it was found that PRC2 binds similarly to both D- and L-G4 RNAs, suggesting that these interactions are independent of stereochemistry. Moreover, herein it is shown that D- and L-RNAs bind to the same site on PRC2, enabling L-G4 RNAs to outcompete native substrates for binding. This work challenges the prevailing assumption that L-oligonucleotides are “invisible” to native biology and provides a unique opportunity to develop a novel class of PRC2 inhibitors based nuclease-resistant L-RNA.

The polycomb repressive complex 2 (PRC2) consists of three core subunits, SUZ12, EED, and EZH2, and is responsible for catalyzing the trimethylation of histone H3 lysine 27 (H3K27me³), an epigenetic mark associated with gene silencing [20]. PRC2 plays an essential role in embryonic development and differentiation [20, 21], and dysregulation of PRC2 along with aberrant H3K27me³ is observed in multiple human cancers [22, 23]. Consequently, substantial efforts have been made to develop PRC2 inhibitors as anti-cancer therapeutics [24-26].

PRC2 is known to bind RNA promiscuously both in vitro and in vivo [27-30], and these interactions have important gene regulatory functions. For example, chromatin bound RNAs may recruit PRC2 to specific genomic sites and direct its methyltransferase activity to the underlying chromatin [31]. Although the molecular basis for these interactions remain unclear, emerging evidence now suggests that the presence of guanine (G)-rich RNA motifs are a key determinant for binding by PRC2. For example, Kaneko et al. showed that poly(G), but not poly(A), was bound by PRC2 in vitro [32]. Moreover, Wang et al. recently reported that PRC2 binds G>C,U>>A in single stranded RNA and has a preference for binding folded G-quadruplex (G4) RNA structures [30]. These in vitro data are consistent with the preferential binding of PRC2 to RNAs containing G-tracts in vivo. Together, these observations motivated the questions as to whether the promiscuity of PRC2 towards G-rich RNAs could be extended to mirror image L-RNA and, specifically, L-G4 RNA structures.

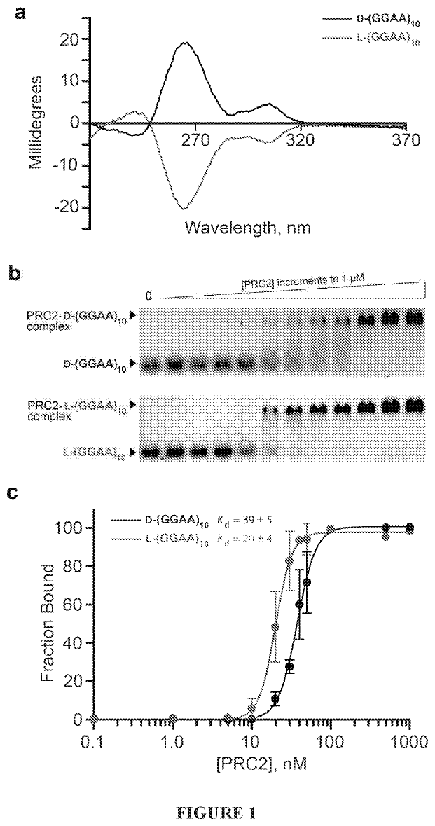

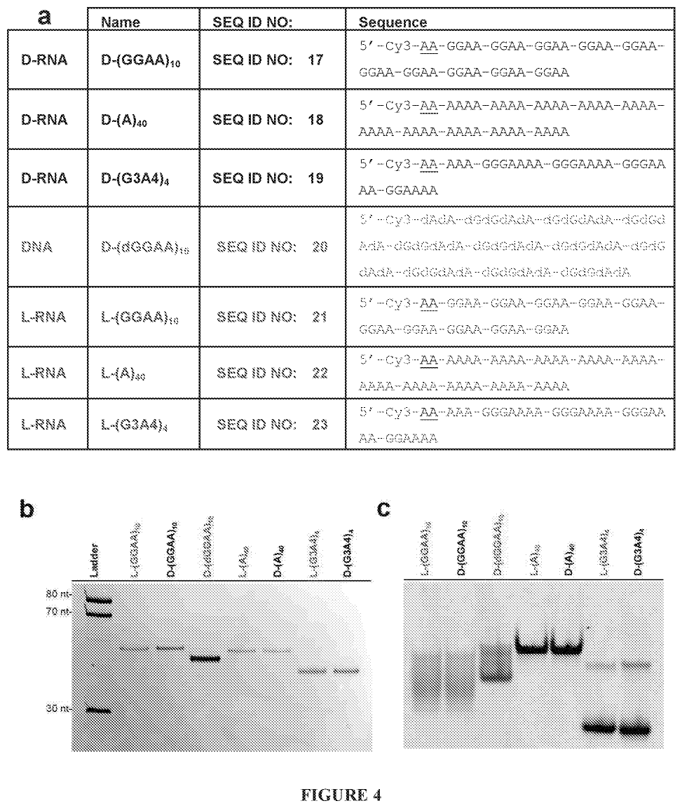

In order to test for potential interactions between PRC2 and L-G4 RNAs, both D- and L-RNA versions of (GGAA)₁₀ were synthesized, a G4-forming RNA previously shown to bind PRC2 with high affinity (K_{d}=7.7±2.4 nM) [30, 33]. For consistency, both enantiomers of (GGAA)₁₀ were Cyanine 3 (Cy3)-labeled at their 5′ ends (FIG. 4a). Formation of G4 structures was confirmed by circular dichroism (CD) spectroscopy for both enantiomers of (GGAA)₁₀ [34] which exhibited the expected mirror symmetry (FIG. 1a) [35]. Folding of these RNAs was further verified by gel electrophoresis (FIG. 4b,c). The ability of each enantiomer of (GGAA)₁₀ to bind PRC2 was then evaluated using an electrophoretic mobility shift assay (EMSA) (FIG. 1b).

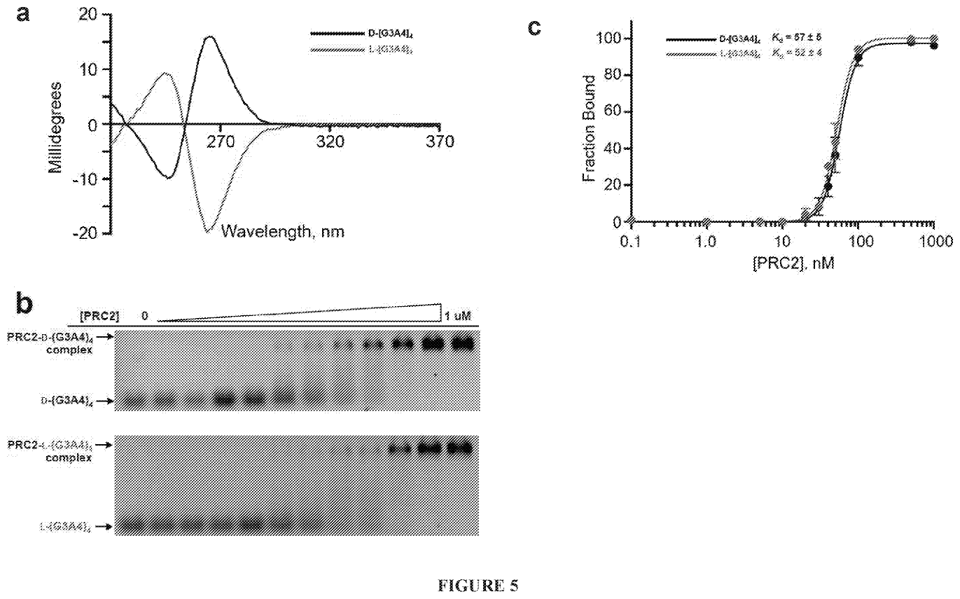

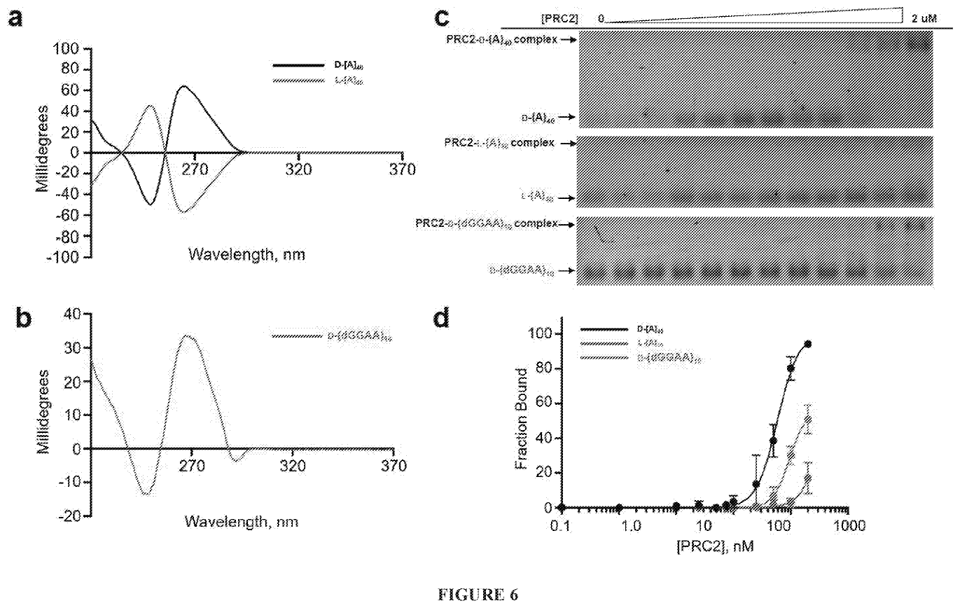

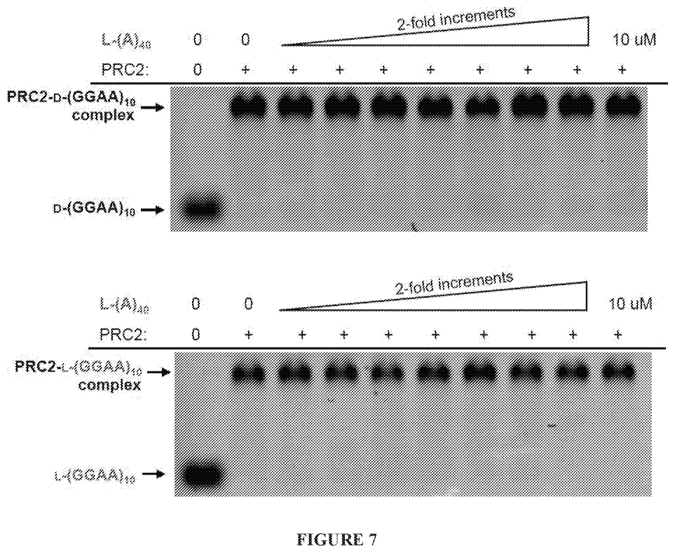

Remarkably, it was found that PRC2 bound with similar affinity to both D- and L-(GGAA)₁₀ (K_{d}=39±5 and 20±4, respectively). Moreover, the Hill coefficients were nearly identical (˜4), suggesting that PRC2 factors bound both enantiomers of (GGAA)₁₀ using a common mode of cooperativity. It is believed that this is the first reported example of a native RNA-binding protein (or protein of any type) recognizing L-RNA. When the K+ cations in the EMSA binding buffer were replaced with Li, which results in destabilization of the G4 structure, the affinity of PRC2 for both D- and L-(GGAA)₁₀ was reduced by 2-fold (FIG. 5). This observation is consistent with Wang et al. [30] and indicates that it is the folded L-G4 structure that is bound to PRC2. To demonstrate that binding of L-G4 RNA by PRC2 was not unique to (GGAA)₁₀, a second G4-forming RNA, (G3A4)₄, was prepared and it was found that it too bound PRC2 irrespective of stereochemistry (K_{d}=57±5 and 52±4 nM for D- and L-(G3A4)₄, respectively) (FIG. 6). In contrast, PRC2 bound weakly to both enantiomers of (A)₄₀ (estimated K_{d}>800 nM; FIG. 7), which is consistent with its preference for G-rich RNA motifs. Interestingly, it was found that the deoxyribose version of D-(GGAA)₁₀, D-(dGGAA), also bound very weakly to PRC2 (estimated K_{d}>1000 nM; FIG. 7). This is despite evidence that D-(dGGAA)₁₀ forms G4 structures. This result was somewhat unexpected given the ability of PRC2 to bind tightly to diverse G-rich sequences, even in the absence of G4 structure formation [30]. Therefore, while the chirality of the sugar moiety is not a determinant for binding of G-rich oligonucleotides by PRC2, the identity of the sugar (ribose versus deoxyribose) is critical.

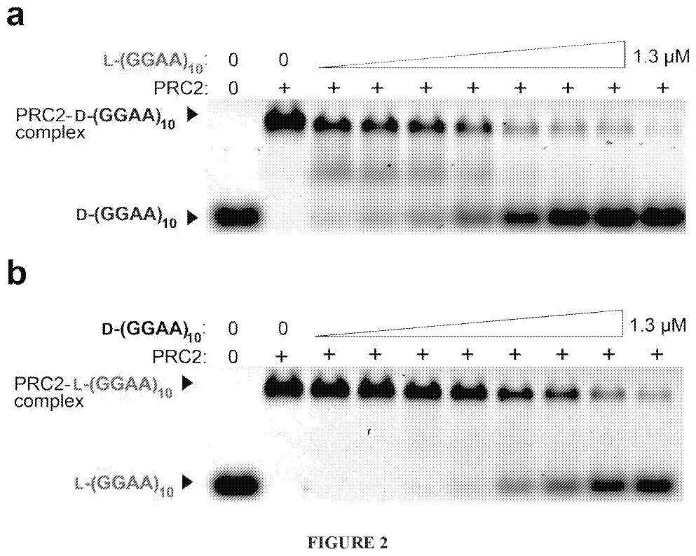

Because D- and L-(GGAA)₁₀ are simply mirror images of each other, it was reasoned that they bind to the same site on PRC2. This may be important because it implies that L-RNA could potentially inhibit PRC2 from binding endogenous D-RNA targets. To test this hypothesis, competition assays were carried out in which increasing concentrations of unlabeled L-(GGAA)₁₀ was added to pre-formed complexes of PRC2 and Cy3-labeled D-(GGAA)₁₀, and vice versa (FIG. 2). These data revealed that both D- and L-(GGAA)₁₀ could outcompete their respective enantiomers for binding to PRC2.

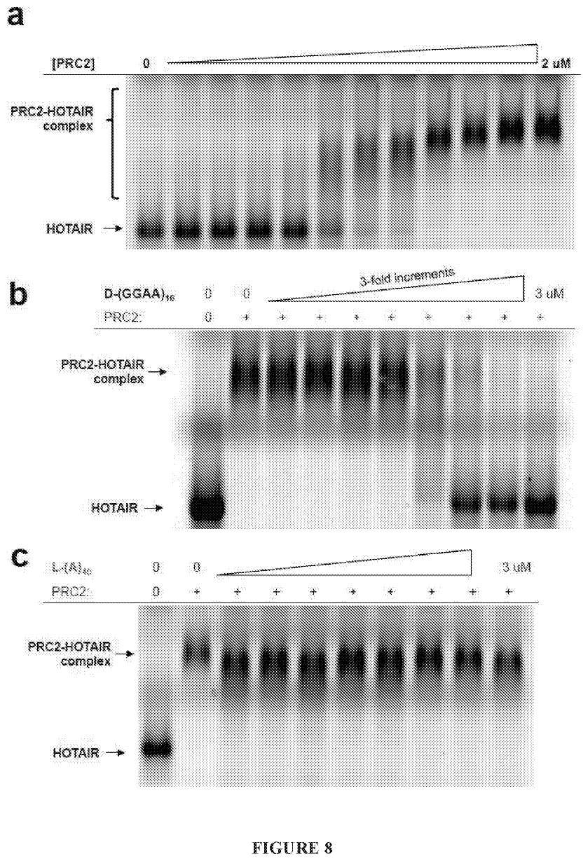

However, the L-(GGAA)₁₀-PRC2 complex was somewhat more resistant to higher concentrations of competitor than its D-RNA counterpart, which may reflect the slightly higher affinity of L-(GGAA)₁₀ for PRC2 as compared to D-(GGAA)₁₀ (FIG. 1). As expected, L-(A)₄₀ failed to compete against both enantiomers of (GGAA)₁₀, even when present in 1,000-fold excess (FIG. 8). Taken together, these data strongly suggest that the same RNA-binding site on PRC2 recognizes both D- and L-(GGAA)₁₀ RNA and, based on their similar binding properties, it is believed that it does so independent of nucleic acid chirality.

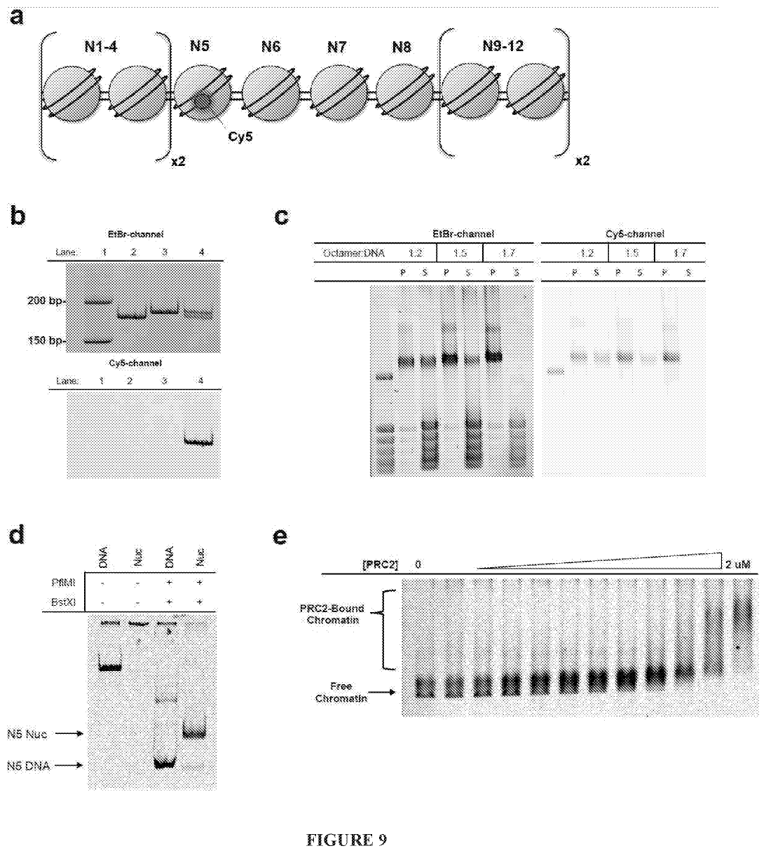

Motivated by the above results, next step was an interrigation of whether L-(GGAA)₁₀ could inhibit PRC2 from binding the long noncoding (lnc)RNA HOTAIR, a bona fide in vivo target required for PRC2 occupancy and H3K27 trimethylation of the HOXD loci [31, 36] and many other genomic sites [37]. HOTAIR is also overexpressed in numerous human cancers and has been shown to promote breast cancer invasiveness and metastasis in a manner that is dependent on PRC2 [38-40]. Thus, disrupting the PRC2-HOTAIR interaction represents a promising approach for developing effective cancer therapy. For these studies, the first 300 nucleotides from the 5′ end of HOTAIR (HOTAIR-300) were employed, which was previously shown to bind PRC2 in vitro (FIG. 9a).

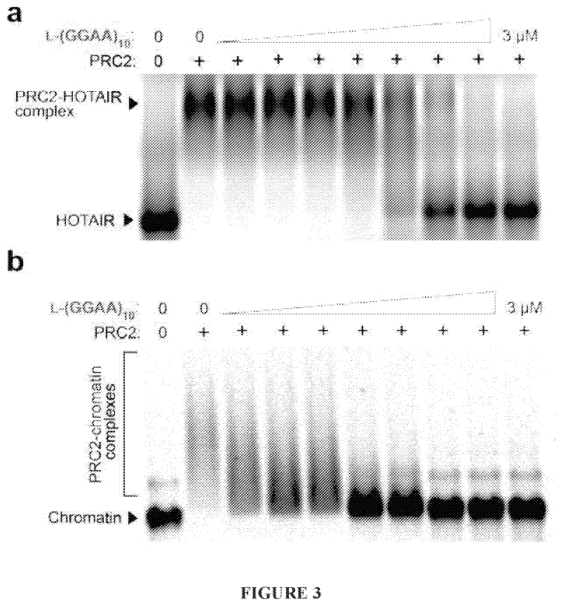

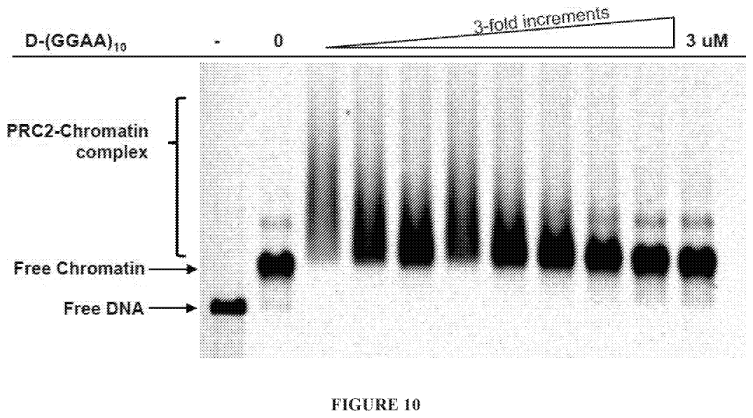

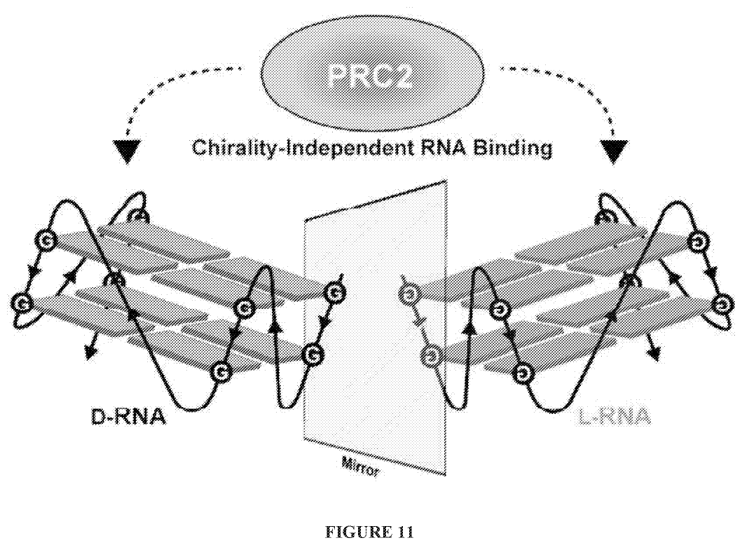

As before, unlabeled L-(GGAA)₁₀ were titrated with pre-formed complexes of PRC2 and Cy5-labeled HOTAIR-300 (FIG. 3a). At a stoichiometric concentration of L-(GGAA)₁₀ relative to PRC2 (250 nM), almost complete dissociation of HOTA1R-300 from PRC2 was observed. Similar results were obtained using D-(GGAA)₁₀, whereas L-poly(A)₄₀ failed to compete (FIG. 9b,c). These results demonstrate that L-(GGAA)₁₀ is an effective inhibitor of lncRNA-PRC2 interactions and further support a common binding site for both D- and L-RNA. Recent studies have shown that native D-RNA, including D-(GGAA)₁₀, is able to disrupt the association of PRC2 with both naked DNA and nucleosomes in vitro, suggesting that RNA and chromatin share the same or mutually exclusive binding sites on PRC2 [33, 41]. In line with these studies, whether L-(GGAA)₁₀ could also prevent PRC2 from binding to chromatin was tested. For these experiments, a Cy5-labeled 12-mer oligonucleosome array reconstituted in vitro using recombinant human histones was employed (FIG. 10). It was found that L-(GGAA)₁₀ was able to disrupt preformed complexes between PRC2 and the oligonucleosome array in a concentration-dependent manner, with no discernable PRC2-chromatin complexes remaining upon the addition of a stoichiometric concentration of L-(GGAA)₁₀ relative to PRC2 (1 μM) (FIG. 3b). Again, these results closely mirrored those obtained using D-(GGAA)₁₀ (FIG. 11). Thus, it was concluded that, like native D-RNA, the interaction of PRC2 with L-RNA and chromatin is mutually antagonistic.

In summary, herein it was demonstrated that PRC2's promiscuous binding to RNA extends to mirror image L-RNA, thereby providing the first evidence that native proteins are capable of recognizing L-oligonucleotides. Remarkably, it was found that PRC2 bound similarly to both enantiomers of G4-forming RNAs, suggesting a chirality-independent mode of recognition. This unexpected and wholly novel finding dramatically broadens the definition of “promiscuous” RNA binding, which now must be expanded to include nucleic acid chirality. Previous studies have shown that native D-RNA is capable of inhibiting PRC2's methyltransferase activity by preventing it from binding its nucleosome substrates [33]. Thus, the present invention discovery that D- and L-RNA bind competitively to the same site on PRC2 opens the door for therapeutic targeting of PRC2 using nuclease-resistant L-G4 RNAs [42-45]. An important next step towards achieving this goal will be to demonstrate that L-G4 RNAs inhibit PRC2 methyltransferase activities in vitro and in human cells.

Importantly, present invnetion discovery that a native RNA-binding protein recognizes L-RNA challenges the prevailing assumption that L-oligonucleotides are “invisible” to the stereospecific environment of the cell and implies that protein interactions should be taken into consideration when designing L-oligonucleotides for intracellular applications. Given the large number of proteins that have been shown to interact with nucleic acids in a nonspecific or “promiscuous” manner [46], it is reasonable to predict that the stereochemical promiscuity observed herein is not unique to PRC2. Therefore, it will be important to undertake future efforts aimed at identifying additional proteins that are capable of interacting with L-RNA (and L-DNA), which if successful will contribute to the future development of intracellular L-oligonucleotide technologies and may ultimately lead to new therapeutic opportunities.

### EXAMPLES

The following examples are provided in order to demonstrate and further illustrate certain preferred embodiments and aspects of the present invention and are not to be construed as limiting the scope thereof.

### Example 1

### Experimental: General Procedures

The DNA and RNA oligonucleotides were either purchased from Integrated DNA Technologies (IDT, Coralville, Iowa) or prepared using an Expedite 8909 DNA/RNA synthesizer. Oligonucleotide synthesis reagents, D-nucleoside phosphoramidites, and Cyanine 3 (Cy3) phosphoramidite were purchased from Glen Research (Sterling, Va.), and L-nucleoside phosphoramidites were purchased from ChemGenes (Wilmington, Mass.). All oligonucleotides were purified by polyacrylamide gel electrophoresis (PAGE) and desalted by ethanol precipitation. Polycomb Repressive Complex 2 was purchased from Active Motif (Carlsbad, Calif.). N-Hydroxysuccinimide (NHS) ester of Cyanine 5 (Cy5) used in the labeling of HOTAIR was acquired from Lumiprobe Life Science Solutions (Hallandale Beach, Fla.).

### Example 2

### Electrophoretic Mobility Shift Assays (EMSA)

Prior to use, Cy3-labeled oligonucleotides (FIG. 4A-C) were diluted to 100 nM in TE Buffer (10 mM TRIS pH 7.5, 1 mM EDTA) and denatured at 95° C. for 10 minutes before being snap cooled on ice for 5 minutes. The oligonucleotides were then diluted to 50 nM in PRC2 binding buffer (50 mM Tris pH 7.4, 100 mM KCl, 2.5 mM MgCl2, 0.1 mM ZnCl2, 2 mM BME, 0.1 mg/mL BSA, and 5% glycerol) and allowed to fold at 37° C. for 30 minutes. In some instances, the KCl was replaced with LiCl (see FIG. 5). The oligonucleotides were then diluted to 2 nM into individual binding reactions (10 μL) containing PRC2 binding buffer and increasing concentrations of PRC2 (0.1-1000 nM). Binding reactions were carried out at 30° C. for 30 minutes and bound and unbound fractions were subsequently separated by 1% agarose gel electrophoresis (0.2×TBE supplemented with 10 mM KOAc or LiOAc as indicated). Agarose gels were run at 4° C. for 75 minutes at 44V. The gels were visualized using GE Typhoon gel imager using the Cy3-emmision filter (excitation: 532 nm; PMT: 950 V) and quantified using ImageQuant TL software.

It was found that the proximity of the Cy3 dye to the terminal guanosines within the G4 RNAs resulted in fluorescent quenching (˜2.5-fold). However, upon PRC2-binding, an increased Cy3 emission was observed that may be attributed to exclusion of the dye from proximal guanosine residues. This phenomenon has been observed previously for G-rich sequences [47, 48]. To account for this phenomenon in calculations, all unbound fluorescent intensities were corrected by a factor equal to the maximum Cy3-signal as measured in the presence of saturating PRC2 divided by the fluorescence of unbound Cy3-RNA.

### Example 3

### Circular Dichroism (CD) Spectroscopy

For CD experiments, oligonucleotides (9.8 μM) were folded as described above in a buffer containing 2 mM sodium phosphate (pH 7.0), 0.1 mM EDTA, and 100 mM of either KCl or LiCl as indicated. Data were obtained from a 450 μL, sample in a quartz cuvette using an Applied Photophysics Chirascan spectrophotometer (Leatherhead, England) at 1 nm intervals from 220 to 370 nm. All data were collected at a constant temperature of 23° C.

### Example 4

### (GGAA)₁₀ Competition Assay

Complexes of PRC2 (100 nM) and Cy3-labeled (GGAA)₁₀ (10 nM) were pre-formed in PRC2 binding buffer as described for EMSAs (30 minutes at 30° C.). Competitive binding experiments were carried out by adding variable concentrations (10-1300 nM) of unlabeled D-(GGAA)₁₀ competitor to the pre-formed PRC2-Cy3-L-(GGAA)₁₀ complexes (or vise versa), and the reaction was allowed to proceed for 30 minutes at 30° C. Bound versus unbound fractions were subsequently separated by 1% agarose gel electrophoresis (0.2×TBE supplemented with 10 mM KOA) and quantified as described above.

### Example 5

### HOTAIR-Binding and Competition Assay

A DNA fragment representing the first 300 nt of HOTAIR (HOTAIR-300) was prepared via PCR assembly using gBlocks Gene Fragments (IDT; Coralville, Iowa). The resulting DNA was added directly into a 100 μL, transcription reaction containing 10 U/μL T7 RNA polymerase, 0.001 U/μL Inorganic pyrophosphatase (IPP), 25 mM MgCl2, 2 mM spermidine, 10 mM DTT, 40 mM Tris (pH 7.9), and 5 mM of each of the four NTPs, where 5-aminoallyl-UTP (Thermo Fisher Scientific, Waltham, Mass.) was supplemented in the transcription reaction at 0.5 mM. The reaction mixture was incubated at 37° C. for 2 hours, then enzymes, DNA, and unincorporated NTPs were removed using a Quick-RNA Mini Prep Plus Kit (Zymo Research, Irvine, Calif.) and pure HOTAIR RNA was obtained in 1× TE buffer. The internally positioned amine functional groups (on the 5-aminoallyl-UTP) were then used to couple a Cy5 NHS-ester (Lumiprobe Life Science Solutions, Hallendale Beach, Fla.) using the provided procedure. For the competition experiments, HOTAIR-PRC2 complexes (25 and 250 nM, respectively) were pre-formed in PRC2 binding buffer as described for EMSAs, and unlabeled (GGAA)₁₀ or (A)₄₀ competitor RNA was added in 3-fold increments from 1 nM to 3 uM. (FIG. 3a and FIG. 9). Bound versus unbound fractions were subsequently separated by 1% agarose gel electrophoresis (0.2×TBE supplemented with 10 mM KOA) and quantified as described above.

### Example 6

### Assembly and Reconstitution of Cy5-Labeled Oligonucleosome Arrays

Human histone proteins were expressed and purified as described previously (Banerjee et. al)[49] and the Cy5-labelled nucleosome array was assembled using a recently published “plug and play” approach [49]. Briefly, two internally positioned nicking endonuclease sites (Nt. BstNBI) were utilized within the fifth 601 unit (N5) of the 12×601 array (FIG. 10a-e) to generate two single-stranded breaks flanking a region of 28 nucleotides (nt). The dual-nicked 12×601 array DNA was then mixed with 20-fold excess of a Cy5-labelled (internally) oligonucleotide insert consisting of a sequence identical to the 28 nt fragment generated by the Nt. BstNBI nicking endonuclease. The mixture was then heated at 80° C. for 20 minutes before being cooled to room temperature at −1° C./min. Following the annealing step (˜1 hour), T4 DNA ligase and ATP (2 mM final concentration) were added to the mixture to reseal the nicks and generate an intact DNA strand. The efficiency of the exchange process (nicking, insertion, and ligation) was carefully monitored in order to ensure complete insertion of the modified oligonucleotide (see FIG. 10b). Oligonudeosome reconstitutions were carried out via salt dialysis and the arrays were purified by selective Mg2+-induced precipitation. Nucleosome saturation was confirmed by selective restriction enzyme digestion (FIG. 10d).

### Example 7

### Chromatin Binding and Competition Assay

In order to confirm that PRC2 was capable of binding the Cy5-labled oligonucleosome array, an EMSA was performed using the same conditions described for the (GGAA)₁₀ binding experiments (FIG. 10e). Using 8 nM arrays, it was found that 1:1 PRC2-chromatin complexes were initiated at PRC2 concentrations <100 nM (FIG. 10e). At higher concentrations of PRC2 (>500 nM), non-stoichiometric binding by PRC2 was observed, resulting higher molecular weight complexes that migrated significantly slower than unbound chromatin when analyzed by agarose gel electrophoresis (0.7%). For the competition assay, a concentration of PRC2 that resulted in a clearly visible interaction by gel electrophoresis (1000 nM PRC2) was chosen and generated PRC2-chromatin complexes by incubating PRC2 with the Cy5-labeled array (8 nM) at 30° C. for 30 minutes in PRC2 binding buffer. Unlabeled competitor (GGAA)₁₀ RNA was then added in 3-fold increments from 1 nM up to 3 uM and analyzed the results by 0.7% agarose gel electrophoresis (0.2×TBE, 10 mM KOAc, 44 V, 5 hours) (FIG. 3b & FIG. 11).

Thus, specific compositions and methods of L-oligonucleotide inhibitors of polycomb repressive complex (PRC2) have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

### REFERENCES

### 1. Cui, K. et al. (2009) “Chromatin Signatures in Multipotent Human Hematopoietic Stem Cells Indicate the Fate of Bivalent Genes During Differentiation,” Cell Stem Cell 4(1), 80-93.

### 2. Barski, A. et al. (2007) “High-Resolution Profiling of Histone Methylations in the Human Genome,” Cell 129(4), 823-837.

3. Kirmizis, A. et al. (2004) “Silencing of Human Polycomb Target Genes Is Associated with Methylation of Histone H3 Lys 27,” Genes Dev 18(13), 1592-1605.

### 4. Bernstein, B. E. et al. (2006) “A Bivalent Chromatin Structure Marks Key Developmental Genes in Embryonic Stem Cells,” Cell 125(2), 315-326.

5. Crea, F. et al. (2011) “Pharmacologic Disruption of Polycomb Repressive Complex 2 Inhibits Tumorigenicity and Tumor Progression in Prostate Cancer,” Mol. Cancer 10, 40-40.
6. Holm, K. et al (2012) “Global H3k27 Trimethylation and Ezh2 Abundance in Breast Tumor Subtypes,” Mol. Oncol. 6(5), 494-506.
7. Raman, J. D. et al. (2005) “Increased Expression of the Polycomb Group Gene, Ezh2, in Transitional Cell Carcinoma of the Bladder,” Clin. Cancer Res. 11(24), 8570.
8. Matsukawa, Y. et al. (2006) “Expression of the Enhancer of Zeste Homolog 2 Is Correlated with Poor Prognosis in Human Gastric Cancer,” Cancer Sci. 97(6), 484-491.
9. McCabe, M. T. et al. (2012) “Mutation of A677 in Histone Methyltransferase Ezh2 in Human B-Cell Lymphoma Promotes Hypertrimethylation of Histone H3 on Lysine 27 (H3k27),” Proc. Natl. Acad. Sci. U.S.A. 109(8), 2989-2994.

### 10. Crea, F. et al. (2012) “Ezh2 Inhibition: Targeting the Crossroad of Tumor Invasion and Angiogenesis,” Cancer Metastasis Rev. 31(3-4), 753-761.

11. Yoo, K. H. and Hennighausen, L. (2012) “Ezh2 Methyltransferase and H3k27 Methylation in Breast Cancer,” Int. J. Sci 8(1), 59-65.
12. Chase, A. and Cross, N. C. P. (2011) “Aberrations of Ezh2 in Cancer,” Clin. Cancer Res. 17(9), 2613.
13. Helmling, S. H. et al. (2004) “RNA World,” Eur J. Biochem. 271(s1), 35-58.
14. Vater, A. and Klussmann, S. (2015) “Turning Mirror-Image Oligonucleotides into Drugs: The Evolution of Spiegelmer® Therapeutics,” Drug Discov. Today 20(1), 147-155.
15. Routh, E. D. et al. (2017) “A G-Quadruplex DNA-Affinity Approach for Purification of Enzymatically Active G4 Resolvase,” JoVE(121), e55496.

### 16. Rhodes, D. and Lipps, H. J. (2015) “G-Quadruplexes and Their Regulatory Roles in Biology,” Nucleic Acids Res. 43(18), 8627-8637.

17. Han, H. and Hurley, L. H. (2000) “G-Quadruplex DNA: A Potential Target for Anti-Cancer Drug Design,” Trends Pharmacol. Sci. 21(4), 136-142.

### 18. Simonsson, T. (2001) “G-Quadruplex DNA Structures-Variations on a Theme,” Chem. 382(4), 621-628.

### 19. Wuts, P. G. M. and Greene, T. W. (2006) Greene's Protective Groups in Organic Synthesis, 4th ed., John Wiley & Sons, Inc., Hoboken, N.J.

### 20. Margueron, R. and Reinberg, D. (2011) “The Polycomb Complex PRC2 and Its Mark in Life,” Nature 469(7330), 343-349.

### 21. Schuettengruber, B. and Cavalli, G. (2009) “Recruitment of Polycomb Group Complexes and Their Role in the Dynamic Regulation of Cell Fate Choice,” Development 136(21), 3531.

22. Ernst, T. et al. (2010) “Inactivating Mutations of the Histone Methyltransferase Gene Ezh2 in Myeloid Disorders,” Nat. Genet. 42, 722.
23. Bracken, A. P. and Helin, K. (2009) “Polycomb Group Proteins: Navigators of Lineage Pathways Led Astray in Cancer,” Nat. Rev. Cancer 9, 773.

### 24. Helin, K. and Dhanak, D. (2013) “Chromatin Proteins and Modifications as Drug Targets,” Nature 502, 480.

25. Kim, K. H. and Roberts, C. W. M. (2016) “Targeting Ezh2 in Cancer,” Nat. Med. 22, 128.
26. Qi, W. et al. (2017) “An Allosteric PRC2 Inhibitor Targeting the H3k27me³ Binding Pocket of Eed,” Nat. Chem. Biol. 13, 381.
27. Davidovich, C. et al. (2013) “Promiscuous RNA Binding by Polycomb Repressive Complex 2,” Nat. Struct. Mol. Biol. 20(11), 1250-1257.
28. Davidovich, C. et al. (2015) “Toward a Consensus on the Binding Specificity and Promiscuity of PRC2 for RNA,” Mol. Cell 57(3), 552-558.
29. Beltran, M. et al. (2016) “The Interaction of PRC2 with RNA or Chromatin Is Mutually Antagonistic,” Genome Res. 26(7), 896-907.
30. Wang, X. et al. (2017) “Targeting of Polycomb Repressive Complex 2 to RNA by Short Repeats of Consecutive Guanines,” Mol. Cell 65(6), 1056-1067.e1055.

### 31. Tsai, M.-C. et al. (2010) “Long Noncoding RNA as Modular Scaffold of Histone Modification Complexes,” Science 329(5992), 689-693.

### 32. Kaneko, S. et al. (2014) “Nascent RNA Interaction Keeps PRC2 Activity Poised and in Check,” Genes Dev. 28(18), 1983-1988.

33. Wang, X. et al. (2017) “Molecular Analysis of PRC2 Recruitment to DNA in Chromatin and its Inhibition by RNA,” Nat. Struct. Mol. Biol. 24, 1028.

### 34. Vorlíc̆ková, M. et al. (2012) “Circular Dichroism and Guanine Quadruplexes,” Methods 57(1), 64-75.

35. Nolte, A. et al. (1996) “Mirror-Design of L-Oligonucleotide Ligands Binding to L-Arginine,” Nat. Biotechnol. 14, 1116.

### 36. Rinn, J. L. et al. (2007) “Functional Demarcation of Active and Silent Chromatin Domains in Human Hox Loci by Noncoding RNAs,” Cell 129(7), 1311-1323.

37. Chu, C. et al. (2011) “Genomic Maps of Lincrna Occupancy Reveal Principles of RNA-Chromatin Interactions,” Mol. Cell 44(4), 667-678.

### 38. Gupta, R. A. et al. (2010) “Long Non-Coding RNA Hotair Reprograms Chromatin State to Promote Cancer Metastasis,” Nature 464, 1071.

### 39. Gutschner, T. and Diederichs, S. (2012) “The Hallmarks of Cancer: A Long Non-Coding RNA Point of View,” RNA Biol. 9(6), 703-719.

### 40. Schmitt, A. M. and Chang, H. Y. (2016) “Long Noncoding RNAs in Cancer Pathways,” Cancer Cell 29(4), 452-463.

41. Cifuentes-Rojas, C. et al. (2014) “Regulatory Interactions between RNA and Polycomb Repressive Complex ,”Mol. Cell 55(2), 171-185.
42. Klußmann, S. et al. (1996) “Mirror-Image RNA That Binds D-Adenosine,” Nat. Biotechnol. 14, 1112.

### 43. Hauser, N. C. et al. (2006) “Utilising the Left-Helical Conformation of L-DNA for Analysing Different Marker Types on a Single Universal Microarray Platform,” Nucleic Acids Res. 34(18), 5101-5111.

44. Kabza, A. M. et al. (2017) “Heterochiral DNA Strand-Displacement Circuits,” J. Am. Chem. Soc. 139(49), 17715-17718.
45. Vater, A. and Klussmann, S. (2015) “Turning Mirror-Image Oligonucleotides into Drugs: The Evolution of Spiegelmer® Therapeutics,” Drug Discov. Today 20(1), 147-155.
46. Jankowsky, E. and Harris, M. E. (2015) “Specificity and Nonspecificity in RNA-Protein Interactions,” Nat. Rev. Mol. Cell Biol. 16, 533.

### 47. Nazarenko, I. et al. (2002) “Effect of Primary and Secondary Structure of Oligodeoxyribonucleotides on the Fluorescent Properties of Conjugated Dyes,” Nucleic Acids Res. 30(9), 2089-2195.

### 48. Mergny, J.-L. and Maurizot, J.-C. (2001) “Fluorescence Resonance Energy Transfer as a Probe for G-Quartet Formation by a Telomeric Repeat,” ChemBioChem 2(2), 124-132.

49. Banerjee, D. R. et al. (2018) “Plug-and-Play Approach for Preparing Chromatin Containing Site-Specific DNA Modifications: The Influence of Chromatin Structure on Base Excision Repair,” J. Am. Chem. Soc. 140(26), 8260-8267.

We claim:

1. A method of treatment of a subject with a condition related to aberrant PRC2 methyltransferase activity comprising:
(a) providing:
(i) L-nucleic acid, and
(b) treating said subject with said L-nucleic acid.

2. The method of claim 1, wherein said L-nucleic acid comprises a guanosine-rich L-oligonucleotide.

3. The method of claim 2, wherein said guanosine-rich L-oligonucleotide comprises at least 1 GGN motif.

4. The method of claim 2, wherein said guanosine-rich L-oligonucleotide comprises 4-100 nucleotides.

5. The method of claim 2, wherein said guanosine-rich L-oligonucleotide forms G-quartets.

6. The method of claim 1, wherein said L-nucleic acid further comprises a chemical modification

7. The method of claim 1, wherein said L-nucleic acid comprises β-L-nucleic acid.

8. The method of claim 1, wherein said L-nucleic acid comprises a non-guanosine-rich L-oligonucleotide.

9. The method of claim 1, wherein said L-nucleic acid comprises L-ribose nucleic acid.

10. The method of claim 9, wherein said L-ribose nucleic acid comprises L-[GGAA]₁₀.

11. The method of claim 9, wherein said L-ribose nucleic acid comprises SEQ ID NO: 2.

12. The method of claim 9, wherein said L-ribose nucleic acid comprises L-[G3A4]₄.

13. The method of claim 9, herein said L-ribose nucleic acid comprises SEQ ID NO: 4.

14. The method of claim 9, wherein said L-ribose nucleic acid comprises a G-quadruplex forming L-RNA.

15. The method of claim 1, wherein said L-nucleic acid comprises L-deoxyribose nucleic acid.

16. The method of claim 15, wherein said L-deoxyribose nucleic acid comprises L-[GGAA]₁₀.

17. The method of claim 15, wherein said L-deoxyribose nucleic acid comprises L-[G3A4]₄.

18. The method of claim 15, wherein said L-deoxyribose nucleic acid comprises SEQ ID NO: 6.

19. The method of claim 15, wherein said L-deoxyribose nucleic acid comprises SEQ ID NO: 8.

20. The method of claim 1, wherein said condition related to aberrant PRC2 methyltransferase activity comprises cancer.

21. A method of treating cancer, comprising: a) providing i) a subject with cancer, said cancer overexpressing PRC2 and ii) a composition comprising L-nucleic acids; and b) administering said composition to said subject.

22. The method of claim 21, wherein said cancer exhibits resistance to SAM-competitive inhibitors.

23. The method of claim 21, wherein said L-nucleic acid inhibits PRC2.

24. The method of claim 21, wherein said L-nucleic acid comprises a guanosine-rich L-oligonucleotide.

25. The method of claim 24, wherein said guanosine-rich L-oligonucleotide comprises at least 1 GGN motif.

26. The method of claim 24, wherein said guanosine-rich L-oligonucleotide comprises 4-100 nucleotides.

27. The method of claim 24, wherein said guanosine-rich L-oligonucleotide forms G-quartets.

28. The method of claim 21, wherein said L-nucleic acid further comprises a chemical modification.

29. The method of claim 21, wherein said L-nucleic acid comprises β-L-nucleic acid.

30. The method of claim 21, wherein said L-nucleic acid comprises a non-guanosine-rich L-oligonucleotide.

31. The method of claim 21, wherein said L-nucleic acid comprises L-ribose nucleic acid.

32. The method of claim 31, wherein said L-ribose nucleic acid comprises L-[GGAA]₁₀.

33. The method of claim 31, wherein said L-ribose nucleic acid comprises SEQ ID NO: 2.

34. The method of claim 31, wherein said L-ribose nucleic acid comprises L-[G3A4]₄.

35. The method of claim 31, wherein said L-ribose nucleic acid comprises SEQ ID NO: 4.

36. The method of claim 31, wherein said L-ribose nucleic acid comprises a G-quadruplex forming L-RNA.

37. The method of claim 21, wherein said L-nucleic acid comprises L-deoxyribose nucleic acid.

38. The method of claim 37, wherein said L-deoxyribose nucleic acid comprises L-[GGAA]₁₀.

39. The method of claim 37, wherein said L-deoxyribose nucleic acid comprises L-[G3A4]₄.

40. The method of claim 37, wherein said L-deoxyribose nucleic acid comprises SEQ ID NO: 6.

41. The method of claim 37, wherein said L-deoxyribose nucleic acid comprises SEQ ID NO: 8.

42. A method of screening, comprising a) providing cancer cells ex vivo, said cancer cells overexpressing PRC2 and ii) at least two different L-nucleic acids; and b) testing said at least two different L-nucleic acids for inhibition of PRC2 by exposing said cancer cells to said L-nucleic acids.

43. The method of claim 42, wherein said cancer cells exhibit resistance to SAM-competitive inhibitors.

44. The method of claim 42, wherein at least one of said L-nucleic acids comprises a guanosine-rich L-oligonucleotide.

45. The method of claim 44, wherein said guanosine-rich L-oligonucleotide comprises at least 1 GGN motif.

46. The method of claim 44, wherein said guanosine-rich L-oligonucleotide comprises 4-100 nucleotides.

47. The method of claim 44, wherein said guanosine-rich L-oligonucleotide forms G-quartets.

48. The method of claim 42, wherein at least one of said L-nucleic acids further comprises a chemical modification.

49. The method of claim 42, wherein at least one of said L-nucleic acids comprises β-L-nucleic acid.

50. The method of claim 42, wherein at least one of said L-nucleic acids comprises a non-guanosine-rich L-oligonucleotide.

51. The method of claim 42, wherein at least one of said L-nucleic acids comprises a L-ribose nucleic acid.

52. The method of claim 51, wherein said L-ribose nucleic acid comprises L-[GGAA]₁₀.

53. The method of claim 51, wherein said L-ribose nucleic acid comprises SEQ ID NO: 2.

54. The method of claim 51, wherein said L-ribose nucleic acid comprises L-[G3A4]₄.

55. The method of claim 51, wherein said L-ribose nucleic acid comprises SEQ ID NO: 4.

56. The method of claim 51, wherein said L-ribose nucleic acid comprises a G-quadruplex forming L-RNA.

57. The method of claim 42, wherein at least one of said L-nucleic acids comprises a L-deoxyribose nucleic acid.

58. The method of claim 57, wherein said L-deoxyribose nucleic acid comprises L-[GGAA]₁₀.

59. The method of claim 57, wherein said L-deoxyribose nucleic acid comprises L-[G3A4]₄.

60. The method of claim 57, wherein said L-deoxyribose nucleic acid comprises SEQ ID NO: 6.

61. The method of claim 57, wherein said L-deoxyribose nucleic acid comprises SEQ ID NO: 8.
